(19) United States
(12) Patent Application Publication
(10) Pub. No.: US 2017/0073285 A1
(43) Pub. Date: Mar. 16, 2017

(54) PROCESSES AND APPARATUSES FOR TOLUENE METHYLATION IN AN AROMATICS COMPLEX

(71) Applicant: UOP LLC, Des Plaines, IL (US)

(72) Inventors: Patrick C. Whitchurch, Sleepy Hollow, IL (US); Gregory R. Werba, Arlington Heights, IL (US)

(21) Appl. No.: 14/885,265

(22) Filed: Oct. 16, 2015

Related U.S. Application Data
(60) Provisional application No. 62/216,425, filed on Sep. 10, 2015.

(51) Int. Cl. C07C 6/06; C07C 5/27; C07C 2/64; B01J 19/24

(52) U.S. Cl. CPC C07C 6/06; B01J 19/245; C07C 5/2729; C07C 2/64; B01J 2219/24

(57) ABSTRACT

This present disclosure relates to processes and apparatuses for toluene methylation in an aromatics complex for producing paraxylene. More specifically, the present disclosure relates to processes and apparatuses wherein a toluene methylation zone is integrated within an aromatics complex for producing paraxylene thus allowing no benzene byproduct to be produced. This may be accomplished by incorporating a toluene methylation process into the aromatics complex and recycling the benzene to the transalkylation unit the aromatics complex.

### CROSS-REFERENCE TO RELATED APPLICATION

This application claims priority from Provisional Application No. 62/216,425 filed Sep. 10, 2015, the contents of which are hereby incorporated by reference.

### FIELD

This present disclosure relates to processes and apparatuses for toluene methylation in an aromatics complex for producing paraxylene. More specifically, the present disclosure relates to processes and apparatuses for toluene methylation within an aromatics complex for producing paraxylene where no benzene byproduct is produced.

### BACKGROUND

The xylene isomers are produced in large volumes from petroleum as feedstocks for a variety of important industrial chemicals. The most important of the xylene isomers is para-xylene, the principal feedstock for polyester, which continues to enjoy a high growth rate from large base demand. Ortho-xylene is used to produce phthalic anhydride, which supplies high-volume but relatively mature markets. Meta-xylene is used in lesser but growing volumes for such products as plasticizers, azo dyes and wood preservers. Ethylbenzene generally is present in xylene mixtures and is occasionally recovered for styrene production, but is usually considered a less-desirable component of C₈ aromatics.

Among the aromatic hydrocarbons, the overall importance of xylenes rivals that of benzene as a feedstock for industrial chemicals. Xylenes and benzene are produced from petroleum by reforming naphtha but not in sufficient volume to meet demand, thus conversion of other hydrocarbons is necessary to increase the yield of xylenes and benzene. Often toluene is de-alkylated to produce benzene or selectively disproportionated to yield benzene and C₈ aromatics from which the individual xylene isomers are recovered.

An aromatics complex flow scheme has been disclosed by Meyers in the Handbook of Petroleum Refining Processes, 2d. Edition in 1997 by McGraw-Hill, and is incorporated herein by reference.

Traditional aromatics complexes send toluene to a transalkylation zone to generate desirable xylene isomers via transalkylation of the toluene with A₉₊ components. A₉₊ components are present in both the reformate bottoms and the transalkylation effluent.

Paraxylene is most often produced from a feedstock which has a methyl to phenyl ration of less than 2. As a result, the paraxylene production is limited by the available methyl groups in the feed. In addition, paraxylene production also typically produces benzene as a byproduct. Since paraxylene is more valuable than benzene and the other byproducts produced in an aromatics complex, there is a desire to maximize the paraxylene production from a given amount of feed. There are also cases where a paraxylene producer would prefer to avoid the production of benzene as a byproduct or paraxylene production. However, there are also cases where a paraxylene producer would prefer to limit the production of benzene as a byproduct or paraxylene production by making adjustments.

### SUMMARY

The present subject matter relates to processes and apparatuses for toluene methylation in an aromatics complex for producing paraxylene. More specifically, the present disclosure relates to processes and apparatuses for toluene methylation within an aromatics complex for producing paraxylene where no benzene byproduct is produced. Integrating a toluene methylation process within an aromatics complex has several benefits. First, the integrated process may increase the amount of paraxylene that can be produced form a given amount of reformate. The integrated process may also reduce the amount of reformate required to produce a fixed amount of paraxylene. Second, the integrated process may avoid the production of benzene as a byproduct from the aromatics complex. These two benefits may be accomplished by incorporating a toluene methylation process into the aromatics complex and recycling the benzene to the transalkylation unit the aromatics complex.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### Definitions

As used herein, the term “stream”, “feed”, “product”, “part” or “portion” can include various hydrocarbon molecules, such as straight-chain, branched, or cyclic alkanes, alkenes, alkadienes, and alkynes, and optionally other substances, such as gases, e.g., hydrogen, or impurities, such as heavy metals, and sulfur and nitrogen compounds. Each of the above may also include aromatic and non-aromatic hydrocarbons.

Hydrocarbon molecules may be abbreviated C₁, C₂, C₃, Cn where “n” represents the number of carbon atoms in the one or more hydrocarbon molecules or the abbreviation may be used as an adjective for, e.g., non-aromatics or compounds. Similarly, aromatic compounds may be abbreviated A₆, A₇, A₈, An where “n” represents the number of carbon atoms in the one or more aromatic molecules. Furthermore, a superscript “+” or “−” may be used with an abbreviated one or more hydrocarbons notation, e.g., C₃₊ or C₃₋, which is inclusive of the abbreviated one or more hydrocarbons. As an example, the abbreviation “C₃₊” means one or more hydrocarbon molecules of three or more carbon atoms.

As used herein, the term “zone” can refer to an area including one or more equipment items and/or one or more sub-zones. Equipment items can include, but are not limited to, one or more reactors or reactor vessels, separation vessels, distillation towers, heaters, exchangers, pipes, pumps, compressors, and controllers. Additionally, an equipment item, such as a reactor, dryer, or vessel, can further include one or more zones or sub-zones.

As used herein, the term “rich” can mean an amount of at least generally 50%, and preferably 70%, by mole, of a compound or class of compounds in a stream.

### BRIEF DESCRIPTION OF THE DRAWINGS

FIG. 1 schematically illustrates an aromatics complex.

FIG. 2 illustrates an aromatics complex having an integrated toluene methylation zone.

Corresponding reference characters indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present disclosure. Also, common but well-understood elements that are useful or necessary in a commercially feasible embodiment are often not depicted in order to facilitate a less obstructed view of these various embodiments of the present disclosure.

### DETAILED DESCRIPTION

The following description is not to be taken in a limiting sense, but is made merely for the purpose of describing the general principles of exemplary aspects. The scope of the present disclosure should be determined with reference to the claims.

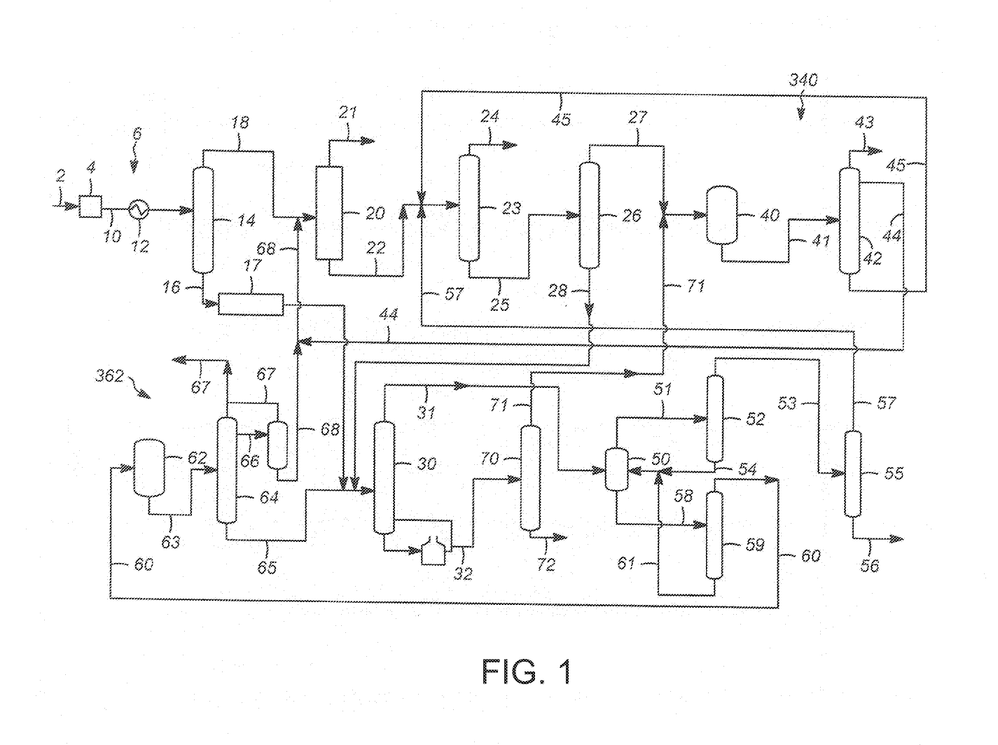

The feedstream to the present process generally comprises alkylaromatic hydrocarbons of the general formula C₆H₍₆₋ₙ₎Rₙ, where n is an integer from 0 to 5 and each R may be CH₃, C₂H₅, C₃H₇, or C₄H₉, in any combination. The aromatics-rich feed stream to the process of the present disclosure may be derived from a variety of sources, including without limitation catalytic reforming, steam pyrolysis of naphtha, distillates or other hydrocarbons to yield light olefins and heavier aromatics-rich byproducts (including gasoline-range material often referred to as “pygas”), and catalytic or thermal cracking of distillates and heavy oils to yield products in the gasoline range. Products from pyrolysis or other cracking operations generally will be hydrotreated according to processes well known in the industry before being charged to the complex in order to remove sulfur, olefins and other compounds which would affect product quality and/or damage catalysts or adsorbents employed therein. Light cycle oil from catalytic cracking also may be beneficially hydrotreated and/or hydrocracked according to known technology to yield products in the gasoline range; the hydrotreating preferably also includes catalytic reforming to yield the aromatics-rich feed stream. FIG. 1 is a simplified flow diagram of an exemplary aromatics-processing complex of the known art directed to the production of at least one xylene isomer. The complex may process an aromatics-rich feed which has been derived, for example, from catalytic reforming in a reforming zone 6. The reforming zone generally includes a reforming unit 4 that receives a feed via conduit 2. The reforming unit typically comprises a reforming catalyst. Usually such a stream will also be treated to remove olefinic compounds and light ends, e.g., butanes and lighter hydrocarbons and preferably pentanes; such removal, however, is not essential to the practice of the broad aspects of this disclosure and is not shown. The aromatics-containing feed stream contains benzene, toluene and C₈ aromatics and typically contains higher aromatics and aliphatic hydrocarbons including naphthenes.

The feed stream is passed via conduit 10 via a heat exchanger 12 to reformate splitter 14 and distilled to separate a stream comprising C₈ and heavier aromatics, withdrawn as a bottoms stream via a bottoms outlet 15 in conduit 16, from toluene and lighter hydrocarbons recovered overhead via conduit 18. The toluene and lighter hydrocarbons are sent to extractive distillation process unit 20 which separates a largely aliphatic raffinate in conduit 21 from a benzene-toluene aromatics stream in conduit 22. The aromatics stream in conduit 22 is separated, along with stripped transalkylation product in conduit 45 and overhead from para-xylene finishing column in conduit 57, in benzene column 23 into a benzene stream in conduit 24 and a toluene-and-heavier aromatics stream in conduit 25 which is sent to a toluene column 26. Toluene is recovered overhead from this column in conduit 27 and may be sent partially or totally to a transalkylation unit 40 as shown and discussed hereinafter.

A bottoms stream from the toluene column 26 is passed via conduit 28, along with bottoms from the reformate splitter in conduit 16, after treating via clay treater 17, and recycle C₈ aromatics in conduit 65, to fractionator 30. The fractionator 30 separates concentrated C₈ aromatics as overhead in conduit 31 from a high-boiling stream comprising C₉, C₁₀ and heavier aromatics as a bottoms stream in conduit 32. This bottoms stream is passed in conduit 32 to heavies column 70. The heavy-aromatics column provides an overhead stream in conduit 71 containing C₉ and at least some of the C₁₀ and C₁₁ aromatics, with higher boiling compounds, primarily higher alkylaromatics, being withdrawn as a bottoms stream via conduit 72.

The C₉₊ aromatics from heavies column in conduit 71 is combined with the toluene-containing overhead contained in conduit 27 as feed to transalkylation reactor 40, which contains a transalkylation catalyst as known in the art to produce a transalkylation product comprising benzene through C₁₁₊ aromatics with xylenes as the focus. The transalkylation product in conduit 41 is stripped in stripper 42 to remove gases in conduit 43 and C₆ and lighter hydrocarbons which are returned via conduit 44 to extractive distillation 20 for recovery of light aromatics and purification of benzene. Bottoms from the stripper are sent in conduit 45 to benzene column 23 to recover benzene product and unconverted toluene.

The C₈-aromatics overhead provided by fractionator 30 contains para-xylene, meta-xylene, ortho-xylene and ethylbenzene and passes via conduit 31 to para-xylene separation process 50. The separation process operates, preferably via adsorption employing a desorbent, to provide a mixture of para-xylene and desorbent via conduit 51 to extract column 52, which separates para-xylene via conduit 53 from returned desorbent in conduit 54; the para-xylene is purified in finishing column 55, yielding a para-xylene product via conduit 56 and light material which is returned to benzene column 23 via conduit 57. A non-equilibrium mixture of C₈-aromatics raffinate and desorbent from separation process 50 is sent via conduit 58 to raffinate column 59, which separates a raffinate for isomerization in conduit 60 from returned desorbent in conduit 61.

The raffinate, comprising a non-equilibrium mixture of xylene isomers and ethylbenzene, is sent via conduit 60 to isomerization reactor 62. The raffinate is isomerized in reactor 62, which contains an isomerization catalyst to provide a product approaching equilibrium concentrations of C₈-aromatic isomers. The product is passed via conduit 63 to deheptanizer 64, which removes C₇ and lighter hydrocarbons with bottoms passing via conduit 65 to xylene column 30 to separate C₉ and heavier materials from the isomerized C₈-aromatics. Overhead liquid from deheptanizer 64 is sent to stripper 66, which removes light materials overhead in conduit 67 from C₆ and C₇ materials which are sent via conduit 68 to the extractive distillation unit 20 for recovery of benzene and toluene values.

There are many possible variations of this scheme within the known art, as the skilled routineer will recognize. For example, the entire C₆-C₈ reformate or only the benzene-containing portion may be subjected to extraction. Para-xylene may be recovered from a C₈-aromatic mixture by crystallization rather than adsorption. Meta-xylene as well as para-xylene may be recovered from a C₈-aromatic mixture by adsorption, and ortho-xylene may be recovered by fractionation. Alternatively, the C₉-and heavier stream or the heavy-aromatics stream is processed using solvent extraction or solvent distillation with a polar solvent or stripping with steam or other media to separate highly condensed aromatics as a residual stream from C₉₊ recycle to transalkylation. In some cases, the entire heavy-aromatic stream may be processed directly in the transalkylation unit. The present disclosure is useful in these and other variants of an aromatics-processing scheme, aspects of which are described in U.S. Pat. No. 6,740,788 which is incorporated herein by reference.

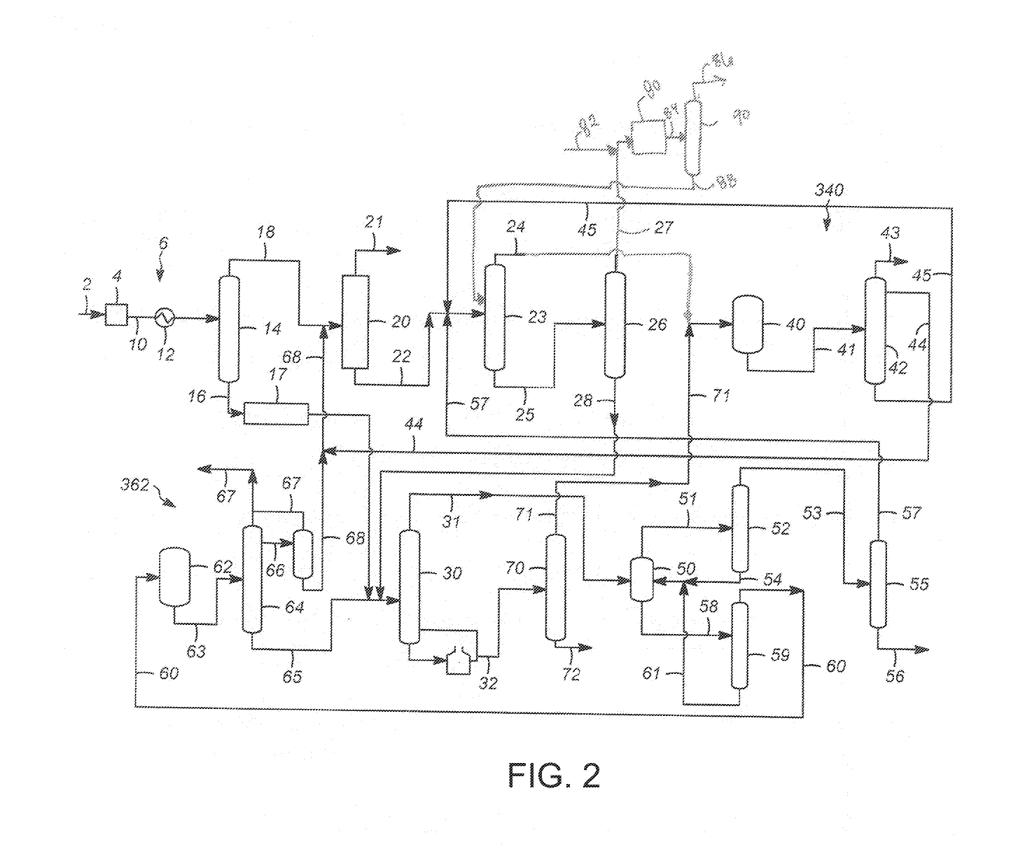

Turning now to FIG. 2, an aromatics complex and process in accordance with one aspect wherein the aromatics complex includes an integrated toluene methylation zone will be illustrated and described. FIG. 2 is a simplified flow diagram of an exemplary aromatics-processing complex of the known art integrated with a toluene methylation unit directed to the production of at least one xylene isomer. The complex may process an aromatics-rich feed which has been derived, for example, from catalytic reforming in a reforming zone 6. The reforming zone generally includes a reforming unit 4 that receives a feed via conduit 2. The reforming unit will typically comprise a reforming catalyst. Usually such a stream will also be treated to remove olefinic compounds and light ends, e.g., butanes and lighter hydrocarbons and preferably pentanes; such removal, however, is not essential to the practice of the broad aspects of this disclosure and is not shown. The aromatics-containing feed stream contains benzene, toluene and C₈ aromatics and typically contains higher aromatics and aliphatic hydrocarbons including naphthenes.

The feed stream is passed via conduit 10 via a heat exchanger 12 to reformate splitter 14 and distilled to separate a stream comprising C₈ and heavier aromatics, withdrawn as a bottoms stream via a bottoms outlet 15 in conduit 16, from toluene and lighter hydrocarbons recovered overhead via conduit 18. The toluene and lighter hydrocarbons are sent to extractive distillation process unit 20 which separates a largely aliphatic raffinate in conduit 21 from a benzene-toluene aromatics stream in conduit 22. The aromatics stream in conduit 22 is separated, along with stripped transalkylation product in conduit 45, an overhead from para-xylene finishing column in conduit 57, and a light aromatic stream in conduit 88 in benzene column 23 into a benzene stream in conduit 24 and a toluene-and-heavier aromatics stream in conduit 25 which is sent to a toluene column 26. The benzene stream in conduit 24 is passed from the benzene column 23 to the transalkylation unit 40. In one embodiment, the transalkylation conditions may include a temperature of about 320° C. to about 440° C. The transalkylation zone may contain a first catalyst. In one embodiment, the first catalyst comprises at least one zeolitic component suitable for transalkylation, at least one zeolitic component suitable for dealkylation and at least one metal component suitable for hydrogenation. Toluene is recovered overhead from this column in conduit 27 and may be sent partially or totally to a toluene methylation unit 80 along with a methanol stream in conduit 82 as shown and discussed hereinafter.

The methanol stream in conduit 82 and the toluene in conduit 27 is passed to the toluene methylation unit 80 and produces a hydrocarbon stream in conduit 84. The hydrocarbon stream in conduit 84 is passed to column 90 which produces an overhead stream in conduit 86 and a bottoms stream in conduit 88. The bottoms stream in conduit 88 is sent back to the benzene column 23. In one embodiment, the toluene methylation product stream has a paraxylene to total xylene ratio of at least about 0.2, or preferably at least about 0.5, or more preferably about 0.8 to 0.95.

The downstream process is the same as in FIG. 1. The C₈-aromatics overhead provided by fractionator 30 contains para-xylene, meta-xylene, ortho-xylene and ethylbenzene and passes via conduit 31 to para-xylene separation process 50. The separation process operates, preferably via adsorption employing a desorbent, to provide a mixture of para-xylene and desorbent via conduit 51 to extract column 52, which separates para-xylene via conduit 53 from returned desorbent in conduit 54; the para-xylene is purified in finishing column 55, yielding a para-xylene product via conduit 56 and light material which is returned to benzene column 23 via conduit 57. A non-equilibrium mixture of C₈-aromatics raffinate and desorbent from separation process 50 is sent via conduit 58 to raffinate column 59, which separates a raffinate for isomerization in conduit 60 from returned desorbent in conduit 61.

The raffinate, comprising a non-equilibrium mixture of xylene isomers and ethylbenzene, is sent via conduit 60 to isomerization reactor 62. The raffinate is isomerized in reactor 62, which contains an isomerization catalyst to provide a product approaching equilibrium concentrations of C₈-aromatic isomers. In one embodiment, the isomerization conditions include a temperature of about 240° C. to about 440° C. Further, the isomerization zone includes s second catalyst. In one embodiment, the second catalyst comprises at least one zeolitic component suitable for xylene isomerization, at least one zeolitic component suitable for ethylbenzene conversion, and at least one metal component suitable for hydrogenation. In one embodiment, the isomerization process is carried out in the vapor phase. In yet another embodiment, the isomerization process is carried out in the liquid phase. In one embodiment, the isomerization process converts ethylbenzene by dealkylation to produce benzene. In another embodiment, the isomerization process converts ethylbenzene by isomerization to produce xylenes.

The product is passed via conduit 63 to deheptanizer 64, which removes C₇ and lighter hydrocarbons with bottoms passing via conduit 65 to xylene column 30 to separate C₉ and heavier materials from the isomerized C₈-aromatics. Overhead liquid from deheptanizer 64 is sent to stripper 66, which removes light materials overhead in conduit 67 from C₆ and C₇ materials which are sent via conduit 68 to the extractive distillation unit 20 for recovery of benzene and toluene values.

There are many possible variations of this scheme within the known art, as the skilled routineer will recognize. For example, the entire C₆-C₈ reformate or only the benzene-containing portion may be subjected to extraction. Para-xylene may be recovered from a C₈-aromatic mixture by crystallization rather than adsorption. The separation zone may also contain a simulated moving bed adsorption unit. In one example, the simulated moving bed adsorption unit uses a desorbent with a lower boiling point than xylenes, such as toluene or benzene. In yet another embodiment, the simulated moving bed adsorption unit uses a desorbent with a higher boiling point than xylenes, such as paradiethylbenzene, paradiisopropylbenzene, tetralin, or paraethyltoluene. Meta-xylene as well as para-xylene may be recovered from a C₈-aromatic mixture by adsorption, and ortho-xylene may be recovered by fractionation. Alternatively, the C₉-and heavier stream or the heavy-aromatics stream is processed using solvent extraction or solvent distillation with a polar solvent or stripping with steam or other media to separate highly condensed aromatics as a residual stream from C₉₊ recycle to transalkylation. In some cases, the entire heavy-aromatic stream may be processed directly in the transalkylation unit. The present disclosure is useful in these and other variants of an aromatics-processing scheme, aspects of which are described in U.S. Pat. No. 6,740,788 which is incorporated herein by reference.

### EXAMPLES

The following examples are intended to further illustrate the subject embodiments. These illustrations of different embodiments are not meant to limit the claims to the particular details of these examples.

TABLE

| | Comparative | | |
|---|---|---|---|
| Case: | Example | Example 1 | Example 2 |
| Toluene Methylation | No | Yes | Yes |
| Included? | | | |
| Xylene Isomerization Type | EB | EB | EB |
| | Dealkylation | Dealkylation | Isomerization |
| Feed Flowrate, | | | |
| MT/yr × 1,000 | | | |
| Reformate | 1703 | 1140 | 1159 |
| Methanol | 0 | 340 | 304 |
| Hydrogen | 8 | 9 | 9 |
| Product Flowrate, | | | |
| MT/yr × 1,000 | | | |
| p-Xylene | 1000 | 1000 | 1000 |
| Benzene | 376 | 0 | 0 |
| Heavy Aromatics | 45 | 42 | 49 |
| Sulfolane Raffinate | 173 | 116 | 123 |
| Water | 0 | 191 | 171 |
| Light Ends | 117 | 141 | 129 |

The Table demonstrates the benefits of having an integrated toluene methylation zone integrated within an aromatics complex. As shown in the Table, Example 1 illustrates an aromatics-processing complex for the production of paraxylene with zero benzene byproduct according to the invention as illustrated in FIG. 2. In this example, the xylene isomerization unit converts ethylbenzene by dealklyation. Further, as shown in the Table, Example 2 illustrates an aromatics-processing complex for the production of paraxylene with zero benzene byproduct according to the invention as illustrated in FIG. 2. In this example, the xylene isomerization unit converts ethylbenzene by isomerization.

It should be noted that various changes and modifications to the presently preferred embodiments described herein will be apparent to those skilled in the art. Such changes and modifications may be made without departing from the spirit and scope of the present subject matter and without diminishing its attendant advantages.

### Specific Embodiments

While the following is described in conjunction with specific embodiments, it will be understood that this description is intended to illustrate and not limit the scope of the preceding description and the appended claims.

A first embodiment of the invention is a process for producing paraxylene with no benzene byproduct, comprising passing a lighter aromatic stream containing benzene and a heavier aromatic stream containing C₉-C₁₀ aromatic compounds to a transalkylation zone; subjecting the lighter aromatic stream and the heavier aromatic stream in the transalkylation zone to transalkylation conditions including the presence of a first catalyst to provide a transalkylation product stream having a greater concentration of toluene to C₈ aromatics; separating by fractionation from the transalkylation product stream a first boiling fraction comprising benzene, a second boiling fraction comprising toluene, a third boiling fraction comprising C₈ aromatics and a fourth boiling fraction comprising C₉₊ aromatics; recycling at least a portion of the benzene from the transalkylation product stream back to the transalkylation zone; passing at least a portion of the second boiling fraction from steps c, g and i and a methanol stream to a toluene methylation zone operating under toluene methylation conditions to produce a toluene methylation product stream; separating by fractionation from the toluene methylation product stream the same fractions described in step c subjecting at least a portion of the third boiling fraction comprising C₈ aromatics of steps c, g and i to a separation zone to selectively remove a para-xylene product and provide a non-equilibrium mixture of C₈ aromatics; subjecting the non-equilibrium mixture of C₈ aromatics to xylene isomerization conditions including the presence of a second catalyst to provide an isomerization product; and separating by fractionation from the isomerization product stream the same fractions described in step c. An embodiment of the invention is one, any or all of prior embodiments in this paragraph up through the first embodiment in this paragraph, wherein the transalkylation conditions include a temperature of about 320° C. to about 440° C.

An embodiment of the invention is one, any or all of prior embodiments in this paragraph up through the first embodiment in this paragraph, wherein the first catalyst comprises at least one zeolitic component suitable for transalkylation, at least one zeolitic component suitable for dealkylation and at least one metal component suitable for hydrogenation. An embodiment of the invention is one, any or all of prior embodiments in this paragraph up through the first embodiment in this paragraph, wherein the toluene methylation product stream has a paraxylene to total xylene ratio of at least about 0.2, or preferably at least about 0.5, or more preferably about 0.8 to 0.95. An embodiment of the invention is one, any or all of prior embodiments in this paragraph up through the first embodiment in this paragraph, wherein the isomerization conditions include a temperature of about 240° C. to about 440° C. An embodiment of the invention is one, any or all of prior embodiments in this paragraph up through the first embodiment in this paragraph, wherein the second catalyst comprises at least one zeolitic component suitable for xylene isomerization, at least one zeolitic component suitable for ethylbenzene conversion, and at least one metal component suitable for hydrogenation. An embodiment of the invention is one, any or all of prior embodiments in this paragraph up through the first embodiment in this paragraph, wherein the isomerization process is carried out in the vapor phase. An embodiment of the invention is one, any or all of prior embodiments in this paragraph up through the first embodiment in this paragraph, wherein the isomerization process converts ethylbenzene by dealkylation to produce benzene. An embodiment of the invention is one, any or all of prior embodiments in this paragraph up through the first embodiment in this paragraph, wherein the isomerization process converts ethylbenzene by isomerization to produce xylenes. An embodiment of the invention is one, any or all of prior embodiments in this paragraph up through the first embodiment in this paragraph, wherein the isomerization process is carried out in the liquid phase.

An embodiment of the invention is one, any or all of prior embodiments in this paragraph up through the first embodiment in this paragraph, wherein all of the benzene is recycled to the transalkylation zone. An embodiment of the invention is one, any or all of prior embodiments in this paragraph up through the first embodiment in this paragraph, wherein the separation zone contains a crystallization unit. An embodiment of the invention is one, any or all of prior embodiments in this paragraph up through the first embodiment in this paragraph, wherein the sepration zone contains a simulated moving bed adsorption unit. An embodiment of the invention is one, any or all of prior embodiments in this paragraph up through the first embodiment in this paragraph, wherein the simulated moving bed adsorption unit uses a desorbent with a lower boiling point than xylenes, such as toluene or benzene. An embodiment of the invention is one, any or all of prior embodiments in this paragraph up through the first embodiment in this paragraph, wherein the simulated moving bed adsorption unit uses a desorbent with a higher boiling point than xylenes, such as paradiethylbenzene, paradiisopropylbenzene, tetralin, or paraethyltoluene.

An embodiment of the invention is one, any or all of prior embodiments in this paragraph up through the first embodiment in this paragraph, further comprising segregating the C₈ aromatic fraction produced in the toluene methylation unit from the other C₈ aromatic fractions produced in the process. An embodiment of the invention is one, any or all of prior embodiments in this paragraph up through the first embodiment in this paragraph, wherein the C₈ aromatic fraction produced in the toluene methylation unit is processed in the same separation zone as one or more other C₈ aromatic fractions, but is introduced at a different feed location An embodiment of the invention is one, any or all of prior embodiments in this paragraph up through the first embodiment in this paragraph, wherein the C₈ aroamtic fraction produced in the toluene methylation unit is processed in a separation zone that is distinct from the separation zone used for the other C₈ aromatic fractions. An embodiment of the invention is one, any or all of prior embodiments in this paragraph up through the first embodiment in this paragraph, wherein the C₈ aromatic fraction produced in the toluene methylation unit is processed in a separation zone that contains a crystallization unit An embodiment of the invention is one, any or all of prior embodiments in this paragraph up through the first embodiment in this paragraph, wherein the C₈ aromatic fraction produced in the toluene methylation unit is processed in a separation zone that contains a simulated moving bed adsorption unit.

A second embodiment of the invention is an apparatus for producing paraxylene, comprising a transalkylation zone in fluid communication with a toluene methylation zone, wherein the toluene methylation zone is in fluid communication with an aromatics separation zone, wherein the aromatics separation zone is in fluid communication with an isomerization zone.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims. In the foregoing, all temperatures are set forth in degrees Celsius and, all parts and percentages are by weight, unless otherwise indicated.

1. A process for producing paraxylene with no benzene byproduct, comprising:
passing a lighter aromatic stream containing benzene and a heavier aromatic stream containing C₉-C₁₀ aromatic compounds to a transalkylation zone;
subjecting the lighter aromatic stream and the heavier aromatic stream in the transalkylation zone to transalkylation conditions including the presence of a first catalyst to provide a transalkylation product stream having a greater concentration of toluene to C₈ aromatics;
separating by fractionation from the transalkylation product stream a first boiling fraction comprising benzene, a second boiling fraction comprising toluene, a third boiling fraction comprising C₈ aromatics and a fourth boiling fraction comprising C₉₊ aromatics;
recycling at least a portion of the benzene from the transalkylation product stream back to the transalkylation zone;
passing at least a portion of the second boiling fraction from steps c, g and i and a methanol stream to a toluene methylation zone operating under toluene methylation conditions to produce a toluene methylation product stream;
separating by fractionation from the toluene methylation product stream the same fractions described in step c
subjecting at least a portion of the third boiling fraction comprising C₈ aromatics of steps c, g and i to a separation zone to selectively remove a para-xylene product and provide a non-equilibrium mixture of C₈ aromatics;
subjecting the non-equilibrium mixture of C₈ aromatics to xylene isomerization conditions including the presence of a second catalyst to provide an isomerization product; and
separating by fractionation from the isomerization product stream the same fractions described in step c.

2. The process according to claim 1, wherein the transalkylation conditions include a temperature of about 320° C. to about 440° C.

3. The process according to claim 1, wherein the first catalyst comprises at least one zeolitic component suitable for transalkylation, at least one zeolitic component suitable for dealkylation and at least one metal component suitable for hydrogenation.

4. The process according to claim 1, wherein the toluene methylation product stream has a paraxylene to total xylene ratio of at least about 0.2, or preferably at least about 0.5, or more preferably about 0.8 to 0.95.

5. The process according to claim 1, wherein the isomerization conditions include a temperature of about 240° C. to about 440° C.

6. The process according to claim 1, wherein the second catalyst comprises at least one zeolitic component suitable for xylene isomerization, at least one zeolitic component suitable for ethylbenzene conversion, and at least one metal component suitable for hydrogenation.

7. The process according to claim 1, wherein the isomerization process is carried out in the vapor phase.

8. The process according to claim 7, wherein the isomerization process converts ethylbenzene by dealkylation to produce benzene.

9. The process according to claim 7, wherein the isomerization process converts ethylbenzene by isomerization to produce xylenes.

10. The process according to claim 1, wherein the isomerization process is carried out in the liquid phase.

11. The process according to claim 1, wherein all of the benzene is recycled to the transalkylation zone.

12. The process according to claim 1, wherein the separation zone contains a crystallization unit.

13. The process according to claim 1, wherein the sepration zone contains a simulated moving bed adsorption unit.

14. The process according to claim 13, wherein the simulated moving bed adsorption unit uses a desorbent with a lower boiling point than xylenes, such as toluene or benzene.

15. The process according to claim 13, wherein the simulated moving bed adsorption unit uses a desorbent with a higher boiling point than xylenes, such as paradiethylbenzene, paradiisopropylbenzene, tetralin, or paraethyltoluene.

16. The process according to claim 1, further comprising segregating the C₈ aromatic fraction produced in the toluene methylation unit from the other C₈ aromatic fractions produced in the process.

17. The process according to claim 16, wherein the C₈ aromatic fraction produced in the toluene methylation unit is processed in the same separation zone as one or more other C₈ aromatic fractions, but is introduced at a different feed location

18. The process according to claim 16, wherein the C₈ aroamtic fraction produced in the toluene methylation unit is processed in a separation zone that is distinct from the separation zone used for the other C₈ aromatic fractions.

19. The process according to claim 18, wherein the C₈ aromatic fraction produced in the toluene methylation unit is processed in a separation zone that contains a crystallization unit

20. The process according to claim 18, wherein the C₈ aromatic fraction produced in the toluene methylation unit is processed in a separation zone that contains a simulated moving bed adsorption unit

21. An apparatus for producing paraxylene, comprising:
a transalkylation zone in fluid communication with a toluene methylation zone, wherein the toluene methylation zone is in fluid communication with an aromatics separation zone, wherein the aromatics separation zone is in fluid communication with an isomerization zone.
